(19) United States
(12) Patent Application Publication
(10) Pub. No.: US 2024/0384835 A1
(43) Pub. Date: Nov. 21, 2024

(54) CEILING FASTENING APPARATUS FOR MEDICAL DEVICES

(71) Applicant: Amtrion GmbH, Porta Westfalica (DE)

(72) Inventor: Matthias ROSNER, Bad Oeynhausen (DE)

(73) Assignee: Amtrion GmbH, Porta Westfalica (DE)

(21) Appl. No.: 18/666,281

(22) Filed: May 16, 2024

(30) Foreign Application Priority Data
May 16, 2023 (DE) 20 2023 102 647.8

(51) Int. Cl. F16M 13/02

(52) U.S. Cl. CPC F16M 13/027

(57) ABSTRACT

A ceiling fastening apparatus for medical devices, comprising a ceiling-mountable carrier as well as a vertically elongated ceiling tube held on the carrier in a mounting position. The ceiling tube includes a plurality of tube segments. The tube segments are screwed to each other via a thread. Adjacent tube segments provide a screw connection securing element, which is configured to counteract a loosening of the screw connection.

This nonprovisional application claims priority under 35 U.S.C. § 119(a) to German Patent Application No. 20 2023 102 647.8, which was filed in Germany on May 16, 2023, and which is herein incorporated by reference.

### BACKGROUND OF THE INVENTION

### Field of the Invention

The present invention relates to a ceiling fastening apparatus for medical devices, comprising a ceiling-mountable carrier as well as a ceiling tube, which is vertically elongated and held on the carrier in a mounting position. The invention further relates to an installation arrangement, including a ceiling-mounted ceiling fastening apparatus, as well as the use of the ceiling fastening apparatus for the ceiling-suspended mounting of medical devices.

### Description of the Background Art

Generic ceiling fastening apparatuses are used, for example, in operating rooms, emergency rooms, or other medical treatment rooms for the purpose of installing medical devices suspended from the ceiling and preferably pivotable or adjustable in height. If a ceiling fastening apparatus of this type is newly installed, a site measurement is taken, based on the planning documents or locally, and the length of the ceiling tube is ascertained, depending on the desired position which the medical device is to take later on. The ceiling tube is subsequently manufactured to measure and painted on an order-specific basis and made ready for dispatch together with the other components of the ceiling fastening device. If it is determined later on during the installation of the ceiling fastening apparatus that the length of the provided ceiling tube does not correspond to the specification, or if the conditions on site or the stipulations for suspending the medical device have changed in the meantime, either a new ceiling tube must be procured and the further assembly interrupted or the provided ceiling tube must be shortened on the construction site. However, the paintwork of the ceiling tube produced in the factory is then damaged.

### SUMMARY OF THE INVENTION

It is therefore an object of the present invention to provide an improved ceiling fastening apparatus and an improved installation arrangement.

To achieve the object, the invention is characterized in that the ceiling tube includes a plurality of tube segments, the tube segments being screwed to each other, and adjacent tube segments providing a screw connection securing element, which is configured to counteract a loosening of the screw connection.

An advantage of the invention is that the length of the ceiling tube of the ceiling fastening apparatus to be installed may be variably adjusted or suitably selected locally on the construction site. For this purpose, standardized, industrially manufactured ceiling tube segments of different lengths are combined as needed into a ceiling tube. The length of the ceiling tube may thus be adapted to possibly changed conditions without interrupting the installation and without a damage to the paintwork. The tube segments manufactured in different lengths according to the module principle may thus be manufactured cost-effectively in uniform quality.

To prevent the tube segments forming the ceiling tube from loosening over time, adjacent tube segments are secured via a screw connection securing element. The screw connection securing element is designed in such a way that it counteracts an unintentional loosening of the screw connection when the ceiling fastening apparatus is used as intended. At the same time, it may be provided that the screw connection may be loosened during a disassembly, for example, by actively loosening the screw connection securing element or applying an elevated disassembly force.

A pull cord, for example, may be provided as the screw connection securing element, which is guided through the tube segments and is determined in terms of length in such a way that a loosening of the screw connection is blocked, due to the associated elongation of the ceiling tube. For example, the pull cord may be fixed on the end side on the ceiling tube and/or on the ceiling-mounted carrier of the ceiling fastening apparatus and/or a carrier arm system used to fasten the medical device, which is rotatably or fixedly held on a free end of the ceiling tube. For example, a pin or a screw may be used as the screw connection securing element and fix two adjacent tube segments relative to each other in a form-fitting and/or force-fitting manner.

The thread can be designed as a fine-pitch thread. The design of the thread as a fine-pitch thread already advantageously counteracts the unintentional loosening of the screw connection, due to the increased friction in the thread.

The screw connection securing element can be implemented as an internal screw connection securing element. Within the meaning of the invention, a screw connection securing element is internal if it is integrated into the ceiling tube or the tube segments forming the ceiling tube in such a way that it is arranged so as to not be visible to an external viewer. Due to the internal implementation of the screw connection securing element, a high-quality and attractive visual effect of the ceiling tube is advantageously ensured. The segmented ceiling tube may also not at first glance be distinguished from a classic ceiling tube manufactured to length. Moreover, an accumulation of dirt or other harmful substances in the region of the screw connection securing element is counteracted with the internal arrangement. This is advantageous, in particular, in the usual places of use, in particular in an operating room, in an emergency room, or in another medical treatment room.

A centering surface can be provided on the tube segments, which is configured to arrange adjacent tube segments concentrically to each other. The centering surface is preferably oriented in the circumferential direction of the tube segments and provided, for example, with a circumferential design. The centering surface may be provided by a shaft shoulder provided on an end face of the tube segment.

Contact surfaces, which are oriented substantially in parallel to each other and perpendicularly to a longitudinal center axis of the ceiling tube, may be formed on tube segment end faces opposite each other. The contact surfaces are used, in particular, to orient the tube segments to be screwed in alignment with each other or without an angle offset.

Each tube segment can provide a female thread and a male thread. The male thread is assigned, for example, to a first tube segment end face and the female thread to a second tube segment end face opposite the first tube segment end face. Due to the consistently same shape of the different tube segments, an ability to arbitrarily combine the tube segments may be advantageously ensured without adapter pieces or the like. The assembly of the ceiling tube from the individual tube segments is simplified hereby.

The screw connection securing element can be in operative engagement with the thread. For example, the thread may include an, in particular, metallic clamping part, which establishes a friction fit during the production of the screw connection and counteracts a loosening of the screw connection in this way.

A plastic ring and, in particular, a polyamide ring, may be provided as the screw connection securing element and clamping ring. The plastic ring may be provided, for example, in the female thread or adjacent thereto on the tube segment. During the assembly of adjacent tube segments, the plastic ring then enters into operative engagement with the male thread of the adjacent tube segment and establishes the screw connection securing via the friction fit.

An adhesive can be provided as a screw connection securing element operating in a materially bonded manner. The adhesive may be applied, for example, to the male thread of the tube segment. The application of the adhesive may take place in the factory. In this case, the adhesive may be protected against a drying out, a contamination, and an unintentional adhesion during transport by a protective film to be removed at the construction site. For example, the adhesive may be applied locally at the construction site during the assembly.

It may be provided that two or more screw connection securing elements are provided. The two or more screw connection securing elements may be provided with the same or a different design. For example, an adhesive connection may be combined with a clamping ring.

The tube segments which are screwed to the ceiling tube preferably can have an identical outer diameter.

Further, an installation arrangement is provided that comprises a ceiling-mounted ceiling fastening apparatus, the carrier of the ceiling fastening apparatus being fastened to a ceiling, and a ceiling tube, which is held on the carrier, extends vertically lengthwise. The ceiling tube can include the plurality of tube segments. The tube segments are screwed to each other. Adjacent tube segments provide a screw connection securing element, which counteracts an unintentional loosening of the screw connection.

The ceiling fastening apparatus according to the invention is provided, in particular, for the ceiling-mounted suspension of medical devices, for example in operating rooms, emergency rooms, or other medical treatment rooms. Moreover, a use of the ceiling fastening apparatus may be provided in industry and in skilled trades. For example, a diagnostic terminal in a motor vehicle workshop or a control unit in a manufacturing plant may be suspended with the aid of the ceiling fastening apparatus according to the invention.

Features and details of the ceiling fastening apparatus described according to the invention naturally also apply in connection with the installation arrangement and vice versa. Thus, reference may always be made interchangeably to the disclosure of the individual aspects of the invention. The drawings are used only to clarify the invention based on examples and are not limiting in nature.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a sectional representation of a ceiling fastening apparatus, including a ceiling-mounted carrier and a vertically oriented, segmented ceiling tube;

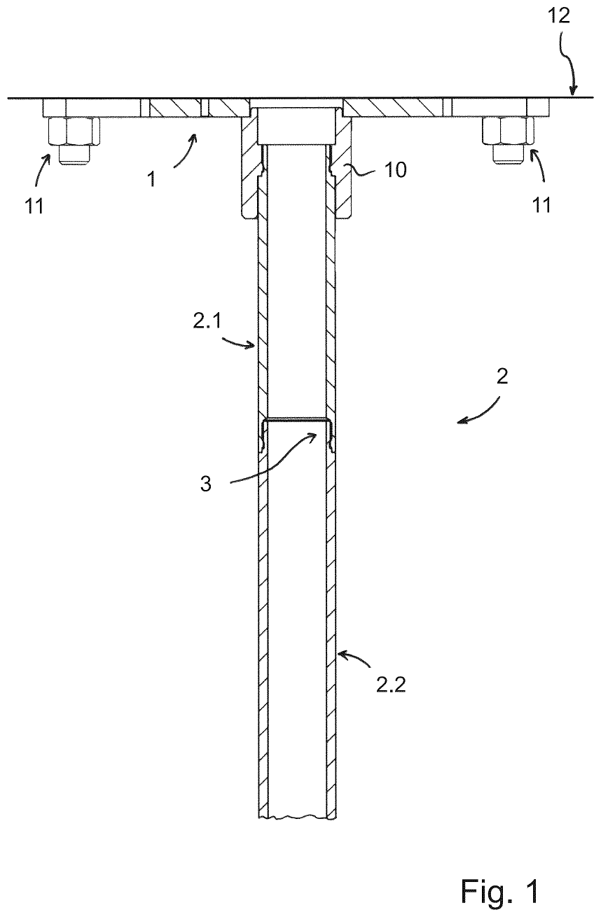

FIG. 2 shows a tube segment of the ceiling tube of the ceiling fastening apparatus according to FIG. 1; and

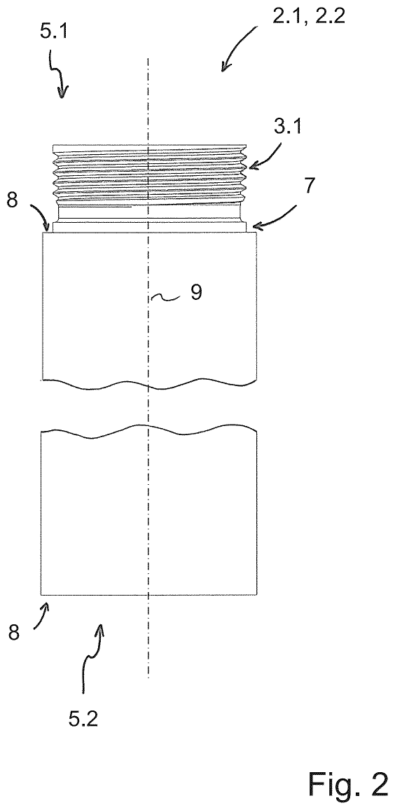

FIG. 3 shows a partial representation of two tube segments of the ceiling tube, including a screw connection securing element, in a premounted situation.

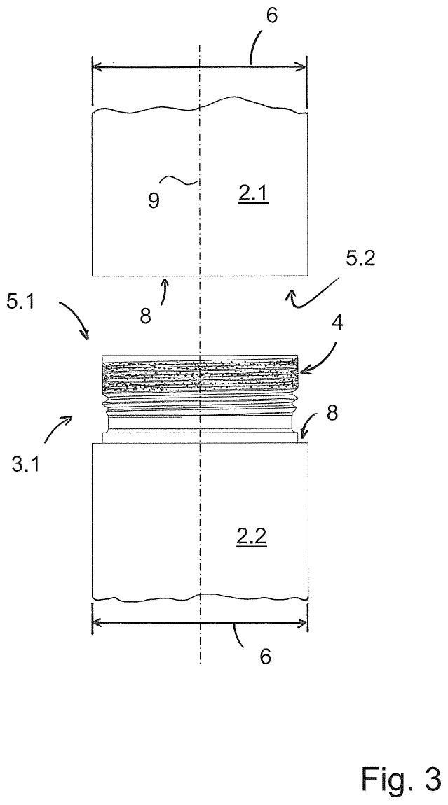

### DETAILED DESCRIPTION

A ceiling fastening apparatus according to the invention as well as an installation arrangement hereby comprises, as essential function components, a carrier 1, which is held on a ceiling 12 via a ceiling screw connection 11, as well as a ceiling tube 2 oriented vertically in an elongated manner in the mounted state. Ceiling tube 2 is fixed to carrier 1 via a tube receptacle 10.

Ceiling tube 2 is implemented as a segmented ceiling tube 2. It comprises a plurality of tube segments 2.1, 2.2, which are screwed to each other via a thread 3. Each tube segment 2.1, 2.2 provides, on opposite tube segment end faces 5.1, 5.2, a male thread 3.1 as well as a female thread, which is designed to correspond to male thread 3.1 and is used to connect adjacent tube segments 2.1, 2.2. Thread 3 is preferably implemented as a fine-pitch thread.

Individual tube segments 2.1, 2.2 may have an identical or a different length. Moreover, an outer diameter 6 of tube segments 2.1, 2.2 is preferably of the same size, so that tube segments 2.1, 2.2 form a ceiling tube 2 having a constant outer diameter 6 in the mounted state.

For assembly purposes, a centering surface 7 extending over a wide area in the direction of longitudinal center axis 9 and running in the circumferential direction is formed on tube segments 2.1, 2.2, as well as an end-face contact surface 8 oriented perpendicularly hereto and with respect to longitudinal center axis 9. Centering surface 7 and contact surface 8, which are oriented perpendicularly to each other, are used for the concentric and plane-parallel contact of adjacent tube segments 2.1, 2.2 with each other. To simplify the assembly, it may be provided that a mounting chamfer is arranged, for example, on centering surface 7 assigned to the female thread, which establishes a self-alignment of tube segments 2.1, 2.2 with each other during the screwing thereof.

To screw the two tube segments 2.1, 2.2 to each other and secure them against an unintentional loosening of the screw connection, an adhesive 4 may be provided as a screw connection securing element of the ceiling fastening apparatus. Adhesive 4 is applied, for example, to male thread 3.1 prior to assembly.

The application of the adhesive may take place in the factory. In this case, it is advantageous to cover adhesive 4 with a protective film. The protective film prevents a drying out of adhesive 4 and an adhesion of foreign bodies to adhesive 4. It is removed immediately prior to assembly. Alternatively, adhesive 4 may be applied as a screw connection securing element to male thread 3.1 immediately prior to mounting tube segments 2.1, 2.2 on the construction site.

The use of adhesive 4 as a screw connection securing element acting in a materially bonded manner is to be understood only as an example. Alternatively, other screw connection securing elements may be used, which counteract an unintentional loosening of the screw connection of adjacent tube segments 2.1, 2.2. For example, a plastic or polyamide ring may be used as the screw connection securing element in the manner of a locknut. For example a metallic clamping part acting upon thread 3 may be used as the screw connection securing element.

Tube segments 2.1, 2.2 which are screwed to ceiling tube 2 may preferably have a different length. This makes it possible to variably adjust the desired absolute length of the ceiling tube as needed by connecting tube segments 2.1, 2.2 of different lengths to each other. The selection of suitable tube segments 2.1, 2.2 may take place locally on the construction side during the mounting of the ceiling fastening apparatus. It is therefore not necessary to take a site measurement or determine the length of the ceiling tube ahead of time, to manufacture a ceiling tube individually manufactured to length for the special application, or to cut an excessively long, provided ceiling tube to measure locally on the construction side.

The invention being thus described, it will be obvious that the same may be varied in many ways. Such variations are not to be regarded as a departure from the spirit and scope of the invention, and all such modifications as would be obvious to one skilled in the art are to be included within the scope of the following claims

What is claimed is:

1. A ceiling fastening apparatus for medical devices, the ceiling fastening apparatus comprising:
a ceiling-mountable carrier; and
a vertically elongated ceiling tube held on the carrier in a mounting position, the ceiling tube comprising a plurality of tube segments that are screwed to each other via a thread, and adjacent tube segments provide a screw connection securing element, which is configured to counteract a loosening of the screw connection.

2. The ceiling fastening apparatus according to claim 1, wherein the thread is a fine-pitch thread.

3. The ceiling fastening apparatus according to claim 1, wherein the screw connection securing element is in operative engagement with the thread.

4. The ceiling fastening apparatus according to claim 1, wherein each tube segment provides a female thread and a male thread, the male thread being assigned to a first tube segment end face, and the female thread being assigned to a second tube segment end face opposite the first tube segment end face.

5. The ceiling fastening apparatus according to claim 1, wherein an adhesive is provided as the screw connection securing element.

6. The ceiling fastening apparatus according to claim 5, wherein the adhesive is applied to the male thread and/or the female thread.

7. The ceiling fastening apparatus according to claim 3, wherein a plastic ring or a polyamide ring is provided as the screw connection securing element.

8. The ceiling fastening apparatus according to claim 1, wherein the screw connection securing element is implemented internally.

9. The ceiling fastening apparatus according to claim 1, wherein a centering surface is provided on the tube segments, the centering surface being configured to arrange adjacent tube segments concentrically to each other, and/or wherein the tube segment end faces have contact surfaces oriented substantially in parallel to each other and substantially perpendicularly to a longitudinal center axis of the ceiling tube.

10. The ceiling fastening apparatus according to claim 1, wherein adjacent tube segments or all tube segments have an identical and/or constant outer diameter, and/or wherein the ceiling tube provides at least two tube segments with a different length.

11. An installation arrangement comprising a ceiling-mounted ceiling fastening apparatus according to claim 1.

12. The ceiling fastening apparatus according to claim 1, wherein the ceiling fastening apparatus is configured for ceiling-mounted suspension of medical devices.
